(19) United States
(12) Patent Application Publication
(10) Pub. No.: US 2025/0346555 A1
(43) Pub. Date: Nov. 13, 2025

(54) METHOD FOR CO-PRODUCING VALPROAMIDE AND SODIUM VALPROATE

(71) Applicants: HUNAN XIANGZHONG PHARMACEUTICAL CO., LTD., Shaoyang, Hunan (CN); HUNAN UNIVERSITY, Changsha, Hunan (CN)

(72) Inventors: Aixi Hu, Changsha, Hunan (CN); Mingfang Li, Changsha, Hunan (CN); Jiao Ye, Changsha, Hunan (CN); Yuyang Liu, Changsha, Hunan (CN)

(21) Appl. No.: 18/866,592

(22) PCT Filed: May 11, 2023
(86) PCT No.: PCT/CN2023/093479

§ 371 Date: Nov. 18, 2024

(30) Foreign Application Priority Data
May 16, 2022 (CN) 202210529577.6

(51) Int. Cl. C07C 231/06; B01J 31/02; C07C 51/41

(52) U.S. Cl. CPC C07C 231/06; B01J 31/0231; C07C 51/412; C07C 2527/06; C07C 2531/02

(57) ABSTRACT

A process for preparing valpromide of formula I and sodium valproate of formula II which comprises: cyanoacetate and 1-chloropropane are subjected to composite catalytic dipropylation in the presence of alkali to obtain 2-cyano-2-valproate of formula III; 2-cyano-2-valproate is hydrolyzed and deacidified to give propylvaleronitrile of formula V; propylvaleronitrile is alcoholized in the presence of acid to give valpromide of formula I and valproate ester of formula VI; and valproate ester is hydrolyzed in a sodium hydroxide solution to afford sodium valproate of formula II.

### TECHNICAL FIELD

The present disclosure relates to a phase-transfer composite catalytic preparation method for 2-cyano-2-valproate and use thereof for co-producing valproic acid (sodium valproate) and valpromide.

### BACKGROUND ART

Sodium valproate and valpromide are both antiepileptic drugs with different treatment ranges. Sodium valproate is a first-choice drug for primary grand mal and absence petit mal, and has a poor therapeutic effect on partial seizures (simple partial, complex partial and partial secondary grand mal seizures). Sodium valproate has a certain therapeutic effect on benign myoclonic epilepsy in infancy and infantile spasm, and is curative for myoclonic absence epileptic seizures with addition of ethosuximide or other antiepileptic drugs. Valpromide is a new antiepileptic drug, and has proved by pharmacological experiments to have an antiepileptic effect twice that of sodium valproate.

Zhou Qiqun et al. [The Synthesis Process Improvement of Sodium Valproate. Chinese Journal of Pharmaceuticals. 1993, 24(8):347-348] disclosed a process using methyl acetoacetate as a starting material to prepare sodium valproate through alkylation catalyzed by quaternary ammonium salt solid-liquid phase transfer, decarboxylation, hydrolysis and salfication:

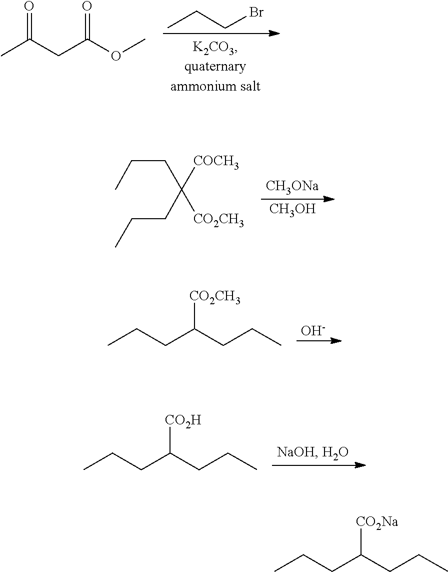

Wang Xueqin et al. [Novel Process for Preparation of Sodium Valproate. Chinese Journal of Pharmaceuticals, 1999, 30(9):389-390] disclosed a process that methyl acetoacetate was condensed with 1-bromopropane in the presence of potassium carbonate and a phase-transfer catalyst of TBAB to afford dipropyl acetoacetate ester, with a yield of 63.1%. In 2019, Lin Fanyou [A Process for Synthesizing Sodium Valproate, CN110563572A, 2019 Dec. 13] also prepared sodium valproate using a phase transfer catalysis of TBAB.

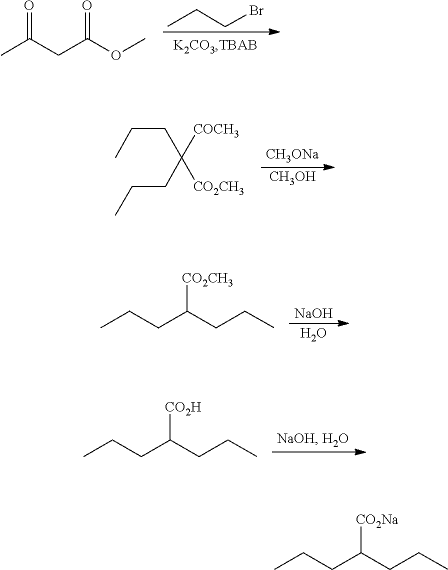

Li Xinyuan, et al. [Synthesis of Antiepileptic Valproic Acid Salts and Amide by Phase Transfer Catalysis. Pharmaceutical Industry, 1984, 5:4-6] and [U.S. Pat. No. 4,127,604] disclosed a process that methyl cyanoacetate was subjected to dipropyl alkylation with 1-bromopropane in the presence of catalysis of quaternary ammonium salt and solid potassium carbonate, and then the product was subjected to hydrolysis, decarboxylation and re-hydrolysis to give valpromide. The valpromide was reacted with nitrous acid to give valproic acid, which was finally salified to give sodium valproate. Nitric oxide and nitrogen dioxide generated by decomposition of nitrous acid pollute environment and corrode equipment.

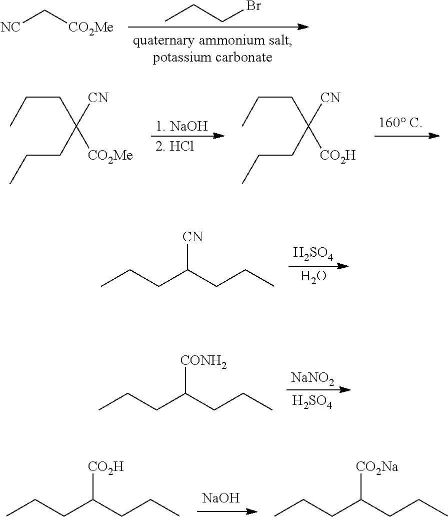

A process of preparing valproic acid from valpromide obtained by decarboxylation and hydrolysis of 2-cyano-2-valproic acid is as follows:

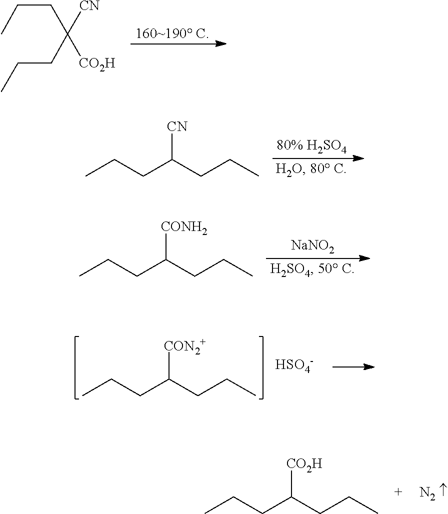

SHANGHAI QINGPING PHARMACEUTICAL CO LTD [Novel Method for Preparing Valproic Acid, CN2021103366414, 2021 Aug. 3; Preparation Method of Sodium Valproate, CN2021103339474, 2021 Aug. 3; Method for Preparing Valproic Acid, CN2021103366274, 2021 Jul. 27] disclosed a one-pot method for preparing valproic acid and sodium valproate using propylvaleronitrile or 2-cyano-2-valproic acid as a starting material. Propylvaleronitrile or 2-cyano-2-valproic acid was hydrolized at 120 to 160° C. for 20-40 hours with an sulfuric acid aqueous solution as a catalyst to give valproic acid with a yield of 70%-80%. The method has a high hydrolysis temperature and long reaction duration. A synthetic route thereof is as follows:

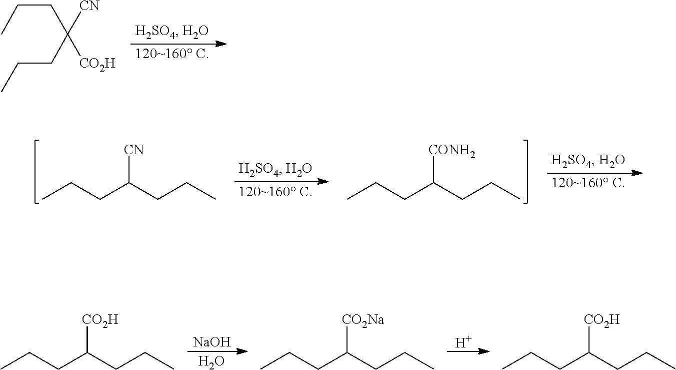

This process employs sulfuric acid to undergo high-temperature decarboxylation and hydrolysis, which may cause side reactions, with a high risk coefficient.

Liu Weiguo [Preparation Method of Sodium Valproate, CN201811564128.5, 2020 Jun. 3] chose ethyl valerate as a raw material to prepare sodium valproate, and a process route is as follows:

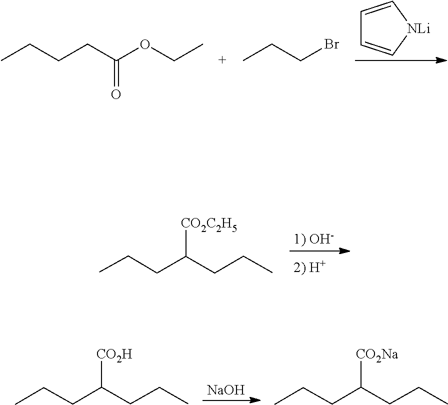

The process for preparing valproic acid of this invention requires very strong base pyrrole lithium salt and low temperature.

### SUMMARY

In one aspect, the present disclosure aims at providing a process for co-producing valpromide of formula I and sodium valproate of formula II, which comprises: cyanoacetate and 1-chloropropane are subjected to composite catalytic dipropylation in the presence of alkali to obtain 2-cyano-2-valproate of formula III; 2-cyano-2-valproate is hydrolyzed and deacidified to give propylvaleronitrile of formula V; propylvaleronitrile is alcoholized in the presence of acid to give valpromide of formula I and valproate ester of formula VI; and valproate ester is hydrolyzed in a sodium hydroxide solution to afford sodium valproate of formula II, and the process thereof is as follows:

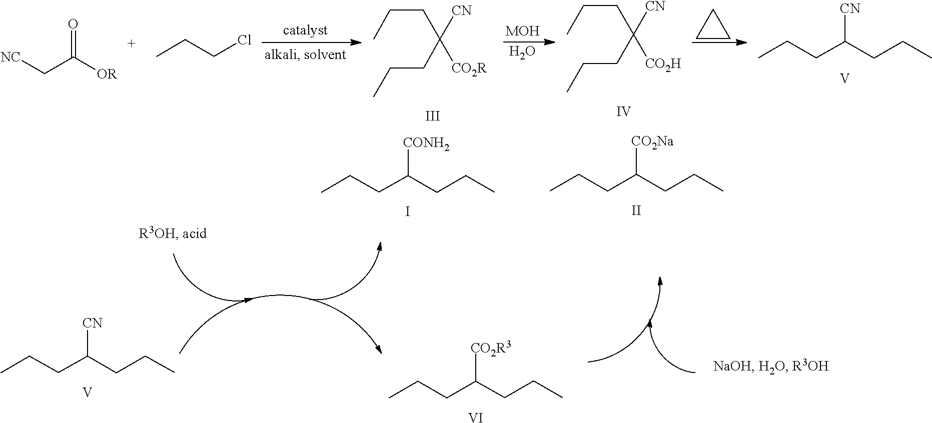

In the above, a preparation method for 2-cyano-2-valproate of formula III is characterized in that cyanoacetate and 1-chloropropane are subjected to composite catalytic dipropylation in the presence of alkali to prepare 2-cyano-2-valproate of formula III, and the preparation reaction thereof is as follows:

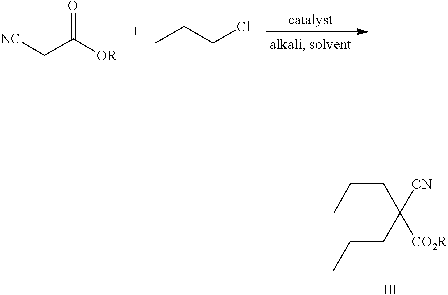

R is methyl or ethyl;
the catalyst consists of catalyst A and catalyst B;
the catalyst A is R₃N, PhNR₂, R₄NX, or R₃R¹NX, wherein R is C1-C4 linear alkyl or C5-C8 linear alkyl; R¹ is PhCH₂, C1-C5 linear alkyl, or C6-C18 linear alkyl; and X is F, Cl, Br, I, or HSO₄; and
the catalyst B is MX, where M is Na, Li, Cs, or K, and X is F, Cl, Br, or I.

R₄NX is tetrabutylammonium chloride, tetrabutylammonium bromide, tetrabutyl-ammonium iodide, tetrabutylammonium hydrogen sulfate, tetraethylammonium fluoride, tetraethylammonium chloride, tetraethylammonium bromide, tetraethylammonium iodide, tetraethylammonium hydrogen sulfate, tetramethylammonium chloride, tetramethylammonium bromide, tetramethylammonium iodide, or tetrapropylammonium bromide.

R₃R¹NX is hexadecyltrimethylammonium bromide, octadecyltrimethylammonium bromide, benzyltriethylammonium chloride, benzyltrimethylammonium chloride, benzyltriethyl-ammonium bromide, hexadecyltriethylammonium bromide, dodecyltriethylammonium bromide, decyltriethylammonium bromide, octyltriethylammonium bromide, hexyltriethylammonium bromide, or trioctylmethylammonium chloride.

R₃N is trimethylamine, triethylamine, tripropylamine, or tributylamine; and PhNR₂ is N,N-dimethylaniline, N,N-diethylaniline, N,N-dipropylaniline, or N,N-dibutylaniline.

MX is, NaBr, KBr, NaI, or KI.

The solvent is one or two selected from the group consisting of THE, DMF, DMC, DMSO, acetonitrile, propionitrile, butyronitrile, 1,4-dioxane, ethylene glycol dimethyl ether, ethylene glycol diethyl ether, diethylene glycol dimethyl ether, diethylene glycol diethyl ether, ethyl acetate and butyl acetate.

The alkali is solid MOH or solid M₂CO₃, wherein M is Na, Li, Cs or K; the solid MOH is granular MOH or powdery MOH; the solid M₂CO₃ is granular M₂CO₃ or powdery M₂CO₃, wherein M is Na, Li, Cs or K.

The powdery M₂CO₃ is 100-mesh M₂CO₃, 150-mesh M₂CO₃, 200-mesh M₂CO₃, 250-mesh M₂CO₃, 300-mesh M₂CO₃, or 350-mesh M₂CO₃, wherein M is Na, Li, Cs or K.

A reaction temperature is in the range of 60-120° C.; and the reaction time is in the range of 1.0-12 h.

Amounts of the catalysts used are in the range of 1:0.01-0.10:0.005-0.05 in a molar ratio of cyanoacetate, the catalyst A, and the catalyst B; wherein cyanoacetate is methyl cyanoacetate, ethyl cyanoacetate, n-propyl cyanoacetate, isopropyl cyanoacetate, n-butyl cyanoacetate, t-butyl cyanoacetate, or benzyl cyanoacetate.

In the above, an alcoholysis method for propylvaleronitrile of formula V is characterized in that propylvaleronitrile reacts with alcohol under acid catalysis to prepare valpromide (solid) of formula I and valproate ester of formula VI, and a preparation reaction thereof is as follows:

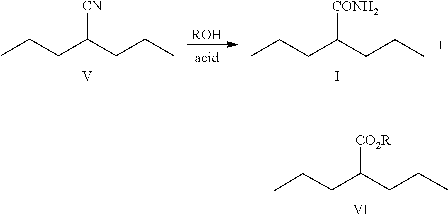

In the above, R is methyl or ethyl; and in an alcoholysis reaction, the acid (acidic material) is HCl (g), AlCl₃, sulfuric acid, thionyl chloride, trifluoromethanesulfonic acid, methanesulfonic acid, benzenesulfonic acid, p-toluenesulfonic acid, or trimethylsilyl trifluoromethanesulfonate.

In the alcoholysis reaction, a molar amount of the acid used is in the range of 1:1.2-4.

In the alcoholysis reaction, mass concentration of the acid is in the range of 30-70%.

A molar amount of alcohol ROH is in the range of propylvaleronitrile:ROH=1:3-8.

In the alcoholysis reaction, an alcoholysis temperature is in the range of 25-100° C.

In the alcoholysis reaction, a duration of the alcoholysis is in the range of 4-24 h.

In the second aspect, the present disclosure provides a method for co-producing valpromide of formula I and sodium valproate of formula II, which is characterized in that propylvaleronitrile of formula V is subjected to alcoholysis and hydrolysis to prepare valpromide and sodium valproate, and a preparation reaction thereof is as follows:

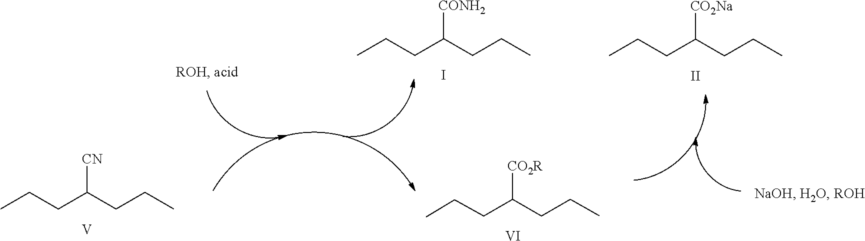

In the above, R is methyl or ethyl.

The acid is HCl (g), AlCl₃, sulfuric acid, thionyl chloride, trifluoromethanesulfonic acid, methanesulfonic acid, benzenesulfonic acid, p-toluenesulfonic acid, or trimethylsilyl trifluoro-methanesulfonate; and a molar ratio of products is in the range of n_{valpromide}:n_{sodium valproate}=1:1.5˜8.0.

In the third aspect, the present disclosure provides a method for co-producing valpromide of formula I and valproic acid of formula VII, which is characterized in that propylvaleronitrile of formula V is subjected to alcoholysis and hydrolysis to prepare valpromide and valproic acid, and a preparation reaction thereof is as follows:

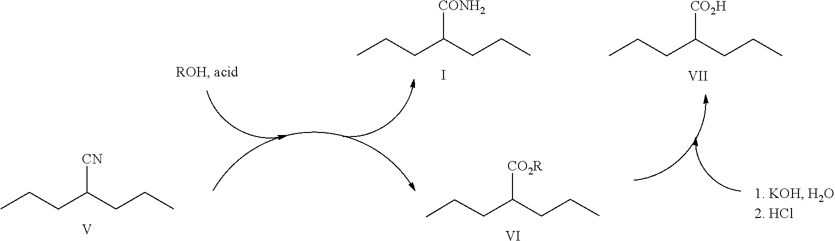

In the above, R is methyl or ethyl.

The acid is HCl (g), AlCl₃, sulfuric acid, thionyl chloride, trifluoromethanesulfonic acid, methanesulfonic acid, benzenesulfonic acid, p-toluenesulfonic acid, or trimethylsilyl trifluoro-methanesulfonate; and a molar ratio of products is in the range of n_{valpromide}:n_{valproic acid}=1:1.5-8.0. Compared with the prior art, the present disclosure has following advantages.

1. In the present disclosure, the composite catalytic dipropylation method of cyanoacetate and 1-chloropropane is used: 1-chloropropane is abundant in supply, rich in source, and inexpensive; the critical dipropylation reaction is complete, providing a guarantee for high quality of a final product. One production route can co-produce two high-quality antiepileptic drugs: sodium valproate and valproamide.

2. In the production process route of the present disclosure, neither the strong base sodium methoxide, sodium ethoxide or potassium tert-butoxide nor costly 1-bromopropane is used; and generation of following by-products is innovatively avoided:

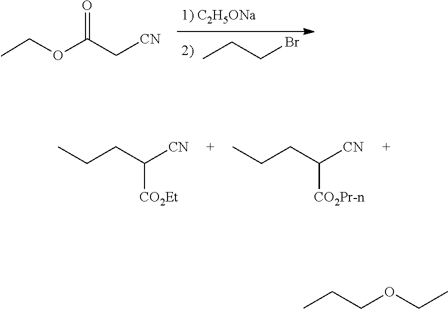

3. Intermediates and products in a production process of the present disclosure are high in purity and easy to separate; according to market requirements, a yield ratio of co-produced bulk drugs valproic acid (sodium valproate) and valpromide can be regulated by controlling reaction conditions of the alcoholysis; investment for production equipment is small, a utilization rate of equipment is high, a production cost is low, and quality is good. The present disclosure has good social and economic benefits.

### DETAILED DESCRIPTION OF EMBODIMENTS

The present disclosure will be further described in detail below in conjunction with examples.

### Example 1

### Preparation of 2-cyano-2-valproic methyl ester

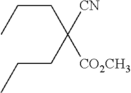

29.73 g (0.30 mol) Of methyl cyanoacetate, 12.0 mmol tetrabutylammonium bromide, 3.0 mmol KI, 91.21 g (0.66 mol) of K₂CO₃, 120 ml of DMF and 58.91 g (0.75 mol) of 1-chloropropane were stirred and reacted at 85° C. for 3.0 h (completion of reaction was monitored by TLC). After reaction was completed, the resultant was slightly cooled and then an inorganic salt was filtered out, and 110 ml of DMF was recovered under a reduced pressure. The inorganic salt was washed with petroleum ether, and the organic phase was washed with water until the aqueous phase become colorless. Then the organic phase was dried over anhydrous sodium sulfate, the sodium sulfate was recovered by suction filtration, and the organic phase was distilled under reduced pressure and dried to give 53.06 g of 2-cyano-2-valproic methyl ester, with a yield of 96.50% (based on methyl cyanoacetate). ¹H NMR (400 MHZ, DMSO-d₆) δ: 3.76 (s, 3H, OCH₃), 1.85-1.75 (m, 4H, CH₂×2), 1.52-1.38 (m, 2H, CH₂), 1.30-1.17 (m, 2H, CH₂), 0.91 (t, J=7.2 Hz, 6H, CH₃×2).

### Example 2

### Preparation of 2-cyano-2-valproic acid

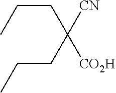

29.73 g (0.30 mol) Of methyl cyanoacetate, 12.0 mmol tetrabutylammonium chloride, 3.0 mmol KI, 91.21 g (0.66 mol) of K₂CO₃, 120 ml of ethylene glycol dimethyl ether and 58.91 g (0.75 mol) of 1-chloropropane were stirred and reacted at 85° C. for 2.5 h (completion of reaction was monitored by TLC). After reaction was completed, the resultant was slightly cooled and then an inorganic salt was filtered out, and 110 ml of ethylene glycol dimethyl ether was recovered under a reduced pressure. The inorganic salt was washed with ethyl acetate, wherein the recovered inorganic salt was KCl and KHCO₃. The organic phase was washed with water until the aqueous phase became colorless. Ethyl acetate was recovered by distillation. Thereafter, 150 ml of 15% KOH was added to the residual yellowish transparent liquid (2-cyano-2-valproic methyl ester), and the mixture was heated and hydrolyzed for 3 h, neutralized by adding concentrated hydrochloric acid to precipitate a solid, which was dried to give 48.69 g of 2-cyano-2-valproic acid as a white solid, with a yield of 95.90% (based on methyl cyanoacetate), and a melting point of 49-50° C. ¹H NMR (400 MHZ, DMSO-d₆) δ: 13.76 (s, 1H, CO₂H), 1.84-1.67 (m, 4H, CH₂×2), 1.54-1.41 (m, 2H, CH₂), 1.36-1.21 (m, 2H, CH₂), 0.92 (t, J=7.2 Hz, 6H, CH₃×2).

### Example 3

### Preparation of 2-cyano-2-valproic acid

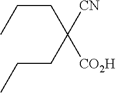

29.73 g (0.30 mol) Of methyl cyanoacetate, 3.33 g (9.0 mmol) of TBAB, 3.0 mmol KI, 91.21 g (0.66 mol) of K₂CO₃, 120 ml of DMF and 58.91 g (0.75 mol) of 1-chloropropane were stirred and reacted at 85° C. for 3.3 h (completion of reaction was monitored by TLC). After the reaction was completed, the resultant was cooled, and an inorganic salt was filtered out, and 110 ml of DMF was recovered under a reduced pressure. The inorganic salt was washed with petroleum ether (100 ml×3), wherein the recovered inorganic salt was KCl and KHCO₃. The organic phase was washed with water (50 ml×3), and the petroleum ether was recovered by distillation to give a yellowish transparent liquid (2-cyano-2-valproate). Into the yellowish transparent liquid 2-cyano-2-valproate, 150 ml of 15% KOH was added. The mixture was hydrolyzed at 65° C. for 3 h, and neutralized in an ice bath by adding concentrated hydrochloric acid to precipitate a white precipitate, which was filtered and dried to give 49.10 g of 2-cyano-2-valproic acid as a white solid, with a yield of 96.72% (based on methyl cyanoacetate) and a melting point of 49-50° C. ¹H NMR (400 MHZ, DMSO-d₆) δ: 13.76 (s, 1H, CO₂H), 1.84-1.67 (m, 4H, CH₂×2), 1.54-1.41 (m, 2H, CH₂), 1.36-1.21 (m, 2H, CH₂), 0.92 (t, J=7.2 Hz, 6H, CH₃×2).

### Example 4

### Preparation of 2-cyano-2-valproic ethyl ester

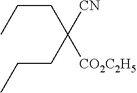

22.6 g (0.20 mol) of ethyl cyanoacetate, 10 mmol TBAC, 5 mmol KI, 60.8 g (0.44 mol) of K₂CO₃ (200 meshes), 80 ml of DMF and 39.3 g (0.50 mol) of 1-chloropropane were stirred and reacted at 80° C. for 6 h. After the reaction was completed, an inorganic salt was filtered out and recovered, and 72 ml of DMF was recovered under a reduced pressure. Thereafter, 100 ml of petroleum ether was added, the organic phase was washed with water until the aqueous phase became colorless, and the organic phase was dried over anhydrous sodium sulfate, followed by suction filtration, rotary evaporation, and drying to give 37.4 g of 2-cyano-2-valproic ethyl ester, with a yield of 94.9% (based on ethyl cyanoacetate). ¹H NMR (400 MHZ, DMSO-d₆) δ: 4.22 (q, J=7.2 Hz, 2H, OCH₂), 1.84-1.75 (m, 4H, CH₂×2), 1.52-1.39 (m, 2H, CH₂), 1.31-1.18 (m, 5H, CH₂+CH₃), 0.91 (t, J=7.2 Hz, 6H, CH₃×2).

### Example 5

### Preparation of 2-cyano-2-valproic acid

22.6 g (0.20 mol) Of ethyl cyanoacetate, 1 mmol TBAC, 60.8 g (0.44 mol) of K₂CO₃ (100 meshes), 20 ml of DMF, 60 ml of butyl acetate, 5 mmol of potassium bromide, and 39.3 g (0.5 mol) of 1-chloropropane were stirred and reacted at 80° C. for 6 h. The resultant was filtered to recover an inorganic salt, and 52 ml of butyl acetate and 17 ml of DMF were recovered under a reduced pressure. Thereafter, 100 ml of petroleum ether was added, and the organic phase was washed with water until the aqueous phase became colorless. The petroleum ether was recovered by rotary evaporation, and 15 ml of 15% KOH was added to the residual yellowish transparent liquid (2-cyano-2-valproic ethyl ester). The mixture was heated and hydrolyzed for 3 h, and neutralized by adding concentrated hydrochloric acid to precipitate a solid, which was dried to give 3.13 g of 2-cyano-2-valproic acid as a white solid, with a yield of 92.60% (based on ethyl cyanoacetate), and a melting point of 49-50° C. ¹H NMR (400 MHZ, DMSO-d₆) δ: 13.76 (s, 1H, CO₂H), 1.84-1.67 (m, 4H, CH₂×2), 1.54-1.41 (m, 2H, CH₂), 1.36-1.21 (m, 2H, CH₂), 0.92 (t, J=7.2 Hz, 6H, CH₃×2).

### Example 6

### Preparation of 2-cyano-2-valproic methyl ester

29.73 g (0.30 mol) Of methyl cyanoacetate, 15.0 mmol of tributylamine, 91.21 g (0.66 mol) of K₂CO₃ (300 meshes), 120 ml of recovered DMF, 5.0 mmol potassium iodide and 58.91 g (0.75 mol) of 1-chloropropane were stirred and reacted at 85° C. for 4.0 h (completion of reaction was monitored by TLC). After reaction was completed, the resultant was filtered to recover an inorganic salt, which was washed with petroleum ether. Thereafter, the organic phase was washed with water until the aqueous phase became colorless. Then the organic phase was dried over anhydrous sodium sulfate, followed by suction filtration, rotary evaporation, and drying to give 53.46 g of 2-cyano-2-valproic methyl ester, with a yield of 94.36%. ¹H NMR (400 MHZ, DMSO-d₆) δ: 3.76 (s, 3H, OCH₃), 1.85-1.75 (m, 4H, CH₂×2), 1.52-1.38 (m, 2H, CH₂), 1.30-1.17 (m, 2H, CH₂), 0.91 (t, J=7.2 Hz, 6H, CH₃×2).

### Example 7

### Preparation of 2-cyano-2-valproic methyl ester

29.73 g (0.30 mol) Of methyl cyanoacetate, 9.0 mmol of recovered tetrabutylammonium bromide, 91.21 g (0.66 mol) of K₂CO₃ (300 meshes), 120 ml of recovered DMF, 2.0 mmol potassium iodide and 58.91 g (0.75 mol) of 1-chloropropane were stirred and reacted at 85° C. for 3.0 h (completion of reaction was monitored by TLC). After reaction was completed, the resultant was filtered to recover an inorganic salt, which was washed with petroleum ether. Thereafter, the organic phase was washed with water until the aqueous phase became colorless. Then the organic phase was dried over anhydrous sodium sulfate, followed by suction filtration, rotary evaporation, and drying to give 53.46 g of 2-cyano-2-valproic methyl ester, with a yield of 94.36%. ¹H NMR (400 MHZ, DMSO-d₆) δ: 3.76 (s, 3H, OCH₃), 1.85-1.75 (m, 4H, CH₂×2), 1.52-1.38 (m, 2H, CH₂), 1.30-1.17 (m, 2H, CH₂), 0.91 (t, J=7.2 Hz, 6H, CH₃×2).

### Example 8

### Preparation of 2-cyano-2-valproic n-propyl ester

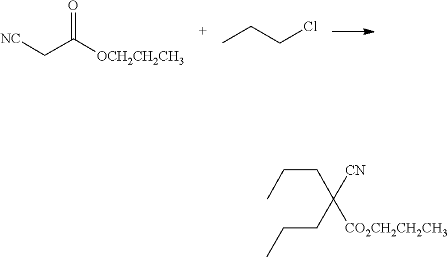

2-Cyano-2-valproic n-propyl ester was prepared from n-propyl cyanoacetate by the method of Example 1.

### Example 9

### Preparation of 2-cyano-2-valproic acid

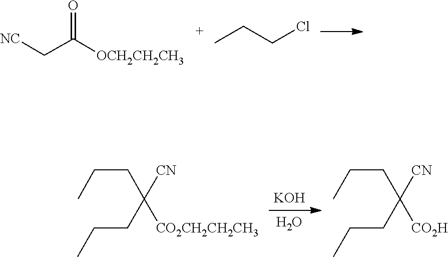

2-Cyano-2-valproic acid was prepared from n-propyl cyanoacetate by the method of Example 2.

### Example 10

### Preparation of 2-cyano-2-valproic isopropyl ester

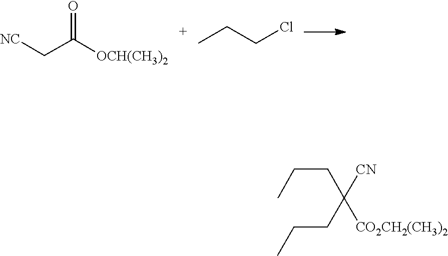

2-Cyano-2-valproic isopropyl ester was prepared from isopropyl cyanoacetate by the method of Example 1.

### Example 11

### Preparation of 2-cyano-2-valproic acid

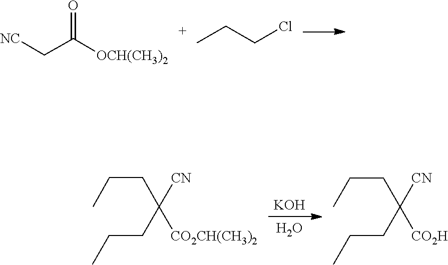

2-Cyano-2-valproic acid was prepared from isopropyl cyanoacetate by the method of Example 2.

### Example 12

### Preparation of Propylvaleronitrile

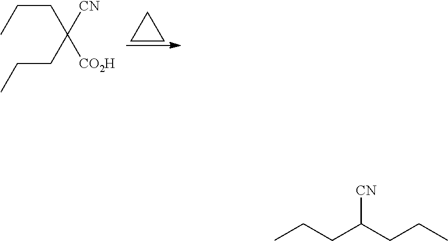

84.6 g of 2-Cyano-2-valproic acid was added into a 250 mL round-bottom flask, heated to 150-155° C., deacidified for 4.0 h, and then subjected to fractional distillation. 165-175° C. fraction was collected to give 55.7 g of propylvaleronitrile as a colorless oil-like substance, with a yield of 89%.

### Example 13

### Preparation of Valpromide and Sodium Valproate

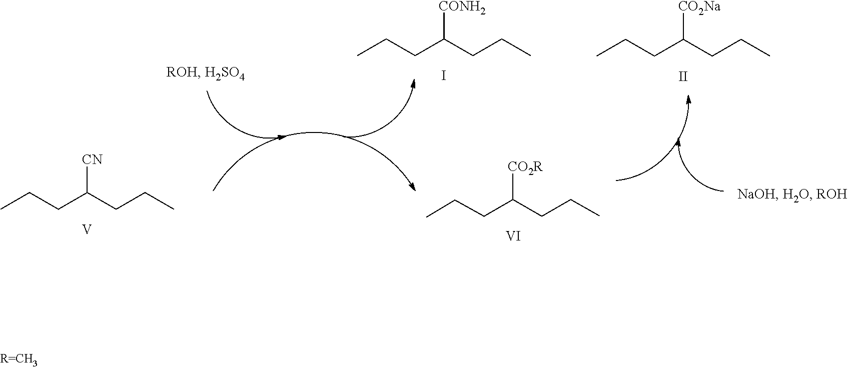

### (1) Preparation of Valpromide

25.04 g of Propylvaleronitrile and 25.63 g of methanol, in an ice bath, were dropwise added with 39.23 g of concentrated sulfuric acid under stirring. The mixture was stirred and reacted at 85° C. for 5 h. Thereafter, 100 ml of water was added, and the mixture was stirred, adjusted to pH 8 with a sodium hydroxide solution. Then the solution was extracted with ethyl acetate, dried over anhydrous sodium sulfate, and was subjected to suction filtration, rotary evaporation, and drying to give a solid-liquid mixture. Then 150 ml of petroleum ether was added and stirred for 0.5 h. The solution was left to stand overnight, followed by suction filtration, and washing with petroleum ether. The filtrate was treated according to step (2). The residue was dried to give 7.04 g of valpromide as a white solid, with a yield of 24.6% (based on propylvaleronitrile), and a melting point of 125.5-126° C. ¹H NMR (400 MHz, DMSO-d₆) δ: 7.26 (s, 1H, CONH₂), 6.71 (s, 1H, CONH₂), 2.18-2.10 (m, 1H, CH), 1.47-1.36 (m, 2H, CH₂), 1.28-1.17 (m, 6H, CH₂+CH₂×2), 0.85 (t, J=5.8 Hz, 6H, CH₃×2).

### (2) Preparation of Sodium Valproate

The filtrate in (1) was subjected to rotary evaporation to recover petroleum ether. 26 g Of sodium hydroxide, 20 ml of water and 20 ml of methanol were added to the residual liquid, and the mixture was heated and refluxed for 5 h. The resultant was cooled, the filtrate was extracted with 50 ml of ethyl acetate (2×25 ml). The organic phase was separated and subjected to rotary evaporation to recover ethyl acetate, and a small amount of residues were propylvaleronitrile, which was recovered. The aqueous phase was concentrated by rotary evaporation and cooled to a room temperature to precipitate a white solid, which was filtered and dried at 50° C. in vacuum to give sodium valproate. Sodium valproate was heated to dissolve with ethyl acetate, and the filtrate was slowly cooled to a room temperature, to precipitate a large amount of white solids, which were filtered and dried at 50° C. in vacuum to give 17.57 g of sodium valproate, with a yield of 52.9% (based on propylvaleronitrile).

### Example 14

### Preparation of Valpromide and Sodium Valproate

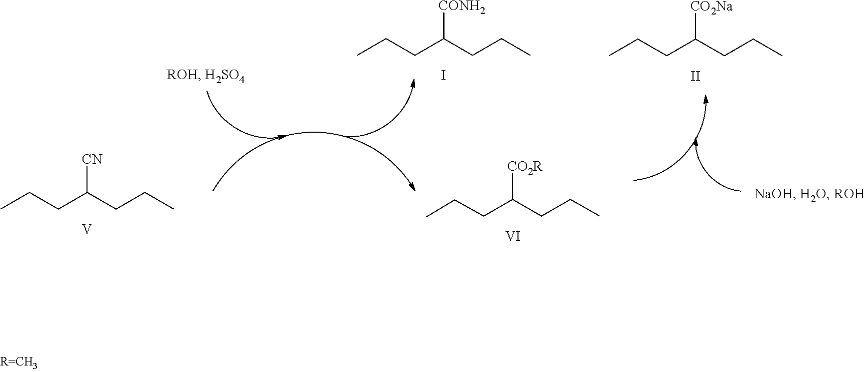

25.04 g Of propylvaleronitrile and 25.63 g of methanol, in an ice bath, were dropwise added with 39.23 g of concentrated sulfuric acid under stirring. The reaction solution was stirred at 75° C. for 10 h, and cooled to a room temperature. Then 100 ml of water was added into the reaction solution with stirring. The mixture was neutralized to pH˜8 with sodium hydroxide solution, and extracted with 150 ml of ethyl acetate (3×50 ml). Then ethyl acetate was recovered by rotary evaporation to give a solid-liquid mixture. 150 ml of petroleum ether was added to precipitate a solid, which was subjected to suction filtration. The filtrate was treated according to step (2). The residue was dried to give 5.4 g of valpromide as a white solid, with a yield of 18.9% (based on propylvaleronitrile), and a melting point of 125.5-126° C. ¹H NMR (400 MHZ, DMSO-d₆) δ: 7.26 (s, 1H, CONH₂), 6.71 (s, 1H, CONH₂), 2.18-2.10 (m, 1H, CH), 1.47-1.36 (m, 2H, CH₂), 1.28-1.17 (m, 6H, CH₂+CH₂×2), 0.85 (t, J=5.8 Hz, 6H, CH₃×2).

The filtrate in (1) was subjected to rotary evaporation to recover petroleum ether. Into the residual liquid, 26 g of sodium hydroxide, 20 ml of water and 20 ml of methanol were added. The mixture was heated and refluxed for 5 h, then the resultant was cooled, and filtrate was extracted with 50 ml of ethyl acetate (2×25 ml). The organic phase was separated, and subjected to rotary evaporation to recover ethyl acetate, and a small amount of residues were propylvaleronitrile, which was recovered. The aqueous phase was concentrated by rotary evaporation and cooled to a room temperature to precipitate a white solid, which was filtered, and dried at 50° C. in vacuum to give sodium valproate. Sodium valproate was heated to dissolve with ethyl acetate, and the filtrate was slowly cooled to a room temperature to precipitate a large amount of white solids, which were filtered and dried at 50° C. in vacuum to give 17.97 g of sodium valproate, with a yield of 54.1% (based on propylvaleronitrile).

### Example 15

### Preparation of Valpromide and Sodium Valproate

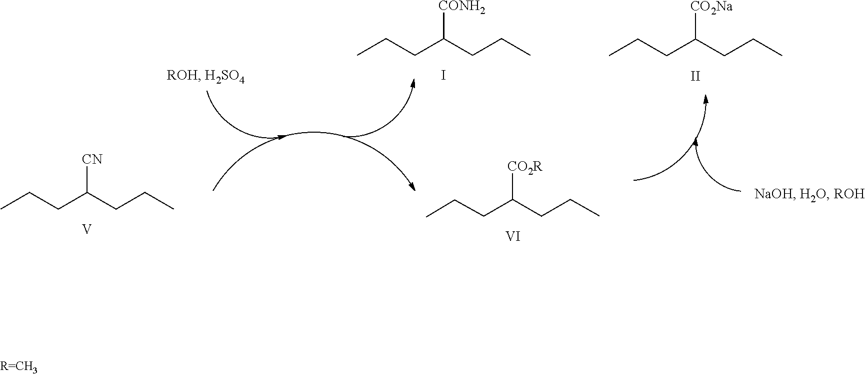

### (1) Preparation of Valpromide

25.04 g Of propylvaleronitrile and 25.63 g of methanol, in an ice bath, were dropwise added with 39.23 g of concentrated sulfuric acid under stirring. The mixture was stirred and reacted at 85° C. for 6 h, then 100 ml of water was added with stirring. The solution was adjusted to pH 8 with a sodium hydroxide solution, extracted with ethyl acetate, dried over anhydrous sodium sulfate, subjected to suction filtration, rotary evaporation, and drying to give a solid-liquid mixture. Thereafter, 150 ml of petroleum ether was added, and mixture was stirred for 0.5 h, left to stand overnight, followed by suction filtration, and washing with petroleum ether. The filtrate was treated according to step (2). The residue was dried to give 6.95 g of valpromide as a white solid, with a yield of 24.3% (based on propylvaleronitrile), and a melting point of 125.5-126° C. ¹H NMR (400 MHZ, DMSO-d₆) δ: 7.26 (s, 1H, CONH₂), 6.71 (s, 1H, CONH₂), 2.18-2.10 (m, 1H, CH), 1.47-1.36 (m, 2H, CH₂), 1.28-1.17 (m, 6H, CH₂+CH₂×2), 0.85 (t, J=5.8 Hz, 6H, CH₃×2)

### (2) Preparation of Sodium Valproate

The filtrate in (1) was subjected to rotary evaporation to recover petroleum ether. Into a residual liquid, 26 g of sodium hydroxide, 20 ml of water and 20 ml of methanol were added. The mixture was heated and refluxed for 5 h. The resultant was cooled, and the aqueous phase was concentrated by rotary evaporation and cooled to room temperature to precipitate a white solid, which was filtered and dried at 50° C. in vacuum to give sodium valproate. Sodium valproate was heated to dissolve with ethyl acetate, the filtrate was slowly cooled to room temperature to precipitate a large amount of white solids, which were filtered and dried at 50° C. in vacuum to give 18.6 g of sodium valproate, with a yield of 56.2% (based on propylvaleronitrile).

### Example 16

### Preparation of Valpromide and Sodium Valproate

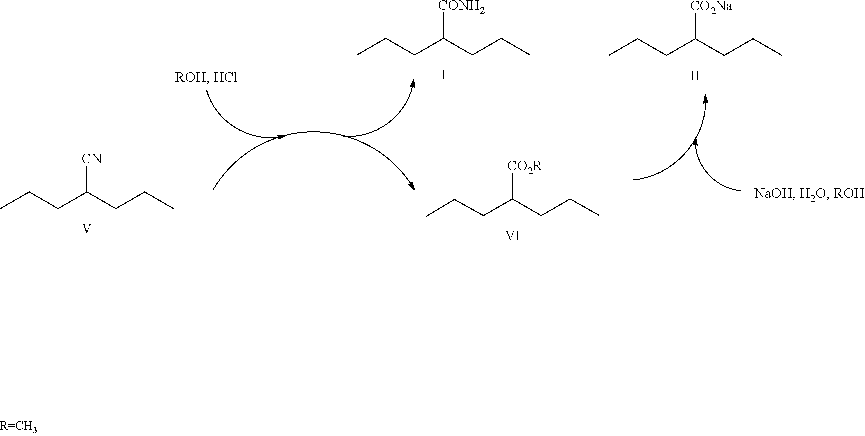

CH₃OH—HCl (g) substituted methanol and sulfuric acid, and according to feed ratios and reaction conditions in Example 15, valpromide and sodium valproate were respectively obtained by the same post-treatment method.

### Example 17

### Preparation of Valpromide and Sodium Valproate

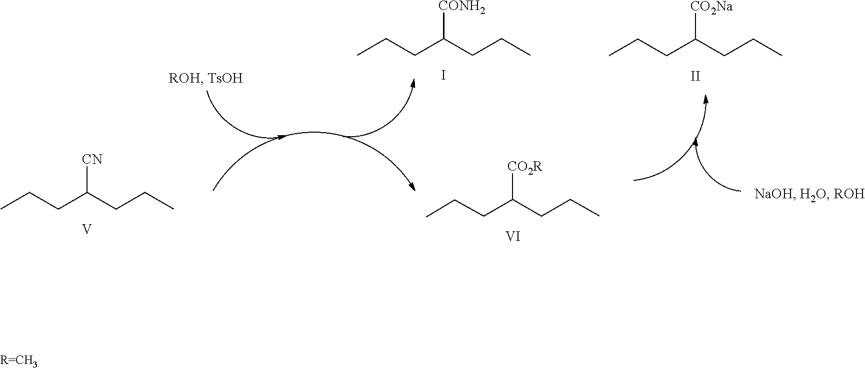

Benzenesulfonic acid or p-toluenesulfonic acid substituted sulfuric acid, and according to feed ratios and reaction conditions in Example 15, valpromide and sodium valproate were respectively obtained by the same post-treatment method.

### Example 18

### Preparation of Valpromide and Sodium Valproate

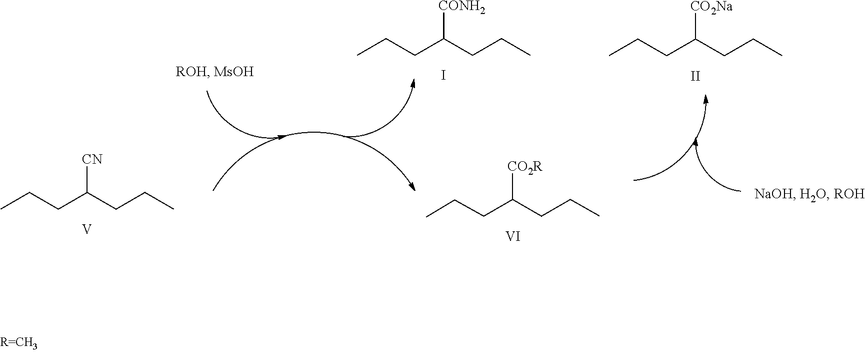

Trifluoromethanesulfonic acid or methanesulfonic acid substituted sulfuric acid, and according to feed ratios and reaction conditions in Example 15, valpromide and sodium valproate were respectively obtained by the same post-treatment method.

### Example 19

### Preparation of Valpromide and Sodium Valproate

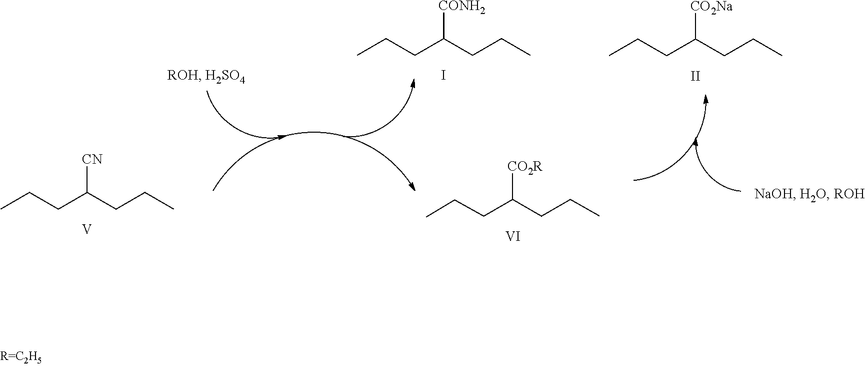

Ethanol was selected to substitute methanol, and according to feed ratios and reaction conditions in Example 15, valpromide and sodium valproate were respectively obtained by the same post-treatment method.

### Example 20

### Preparation of Valpromide and Valproic Acid Methyl Ester

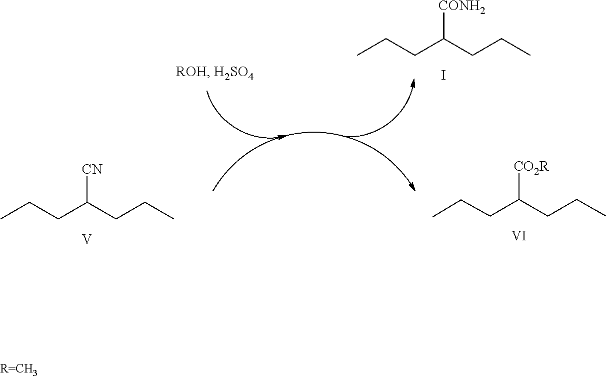

### (1) Preparation of Valpromide

125 g Of propylvaleronitrile and 128 g of methanol, in an ice bath, were dropwise added with 196 g of concentrated sulfuric acid under stirring. The mixture was stirred and reacted at 85° C. for 5.5 h, 500 ml of water was added, and the mixture was stirred, adjusted to pH 8 with a sodium hydroxide solution, extracted with ethyl acetate, dried over anhydrous sodium sulfate, subjected to suction filtration, rotary evaporation, and drying to give a solid-liquid mixture. Thereafter, 700 ml of petroleum ether was added, the mixture was stirred for 0.5 h, left to stand overnight, followed by suction filtration, and washing with petroleum ether. The filtrate was treated according to step (2). The residue was dried to give 35 g of valpromide as a white solid, with a yield of 24.5% (based on propylvaleronitrile), and a melting point of 125.5-126° C. ¹H NMR (400 MHZ, DMSO-d₆) δ: 7.26 (s, 1H, CONH₂), 6.71 (s, 1H, CONH₂), 2.18-2.10 (m, 1H, CH), 1.47-1.36 (m, 2H, CH₂), 1.28-1.17 (m, 6H, CH₂+CH₂×2), 0.85 (t, J=5.8 Hz, 6H, CH₃×2).

### (2) Preparation of Valproic Acid Methyl Ester

Filtrate in (1) was subjected to rotary evaporation to recover petroleum ether, followed by reduced-pressure rectification to give 95 g of valproic acid methyl ester, with a yield of 60.1% (based on propylvaleronitrile). ¹H NMR (400 MHZ, CDCl₃) δ: 3.67 (s, 3H, OCH₃), 2.43-2.32 (m, 1H, CH), 1.65-1.53 (m, 2H, CH₂), 1.47-1.36 (m, 2H, CH₂), 1.33-1.23 (m, 4H, CH₂×2), 0.89 (t, J=7.2 Hz, 6H, CH₃×2).

### Example 21

### Preparation of Valpromide and Valproic Acid

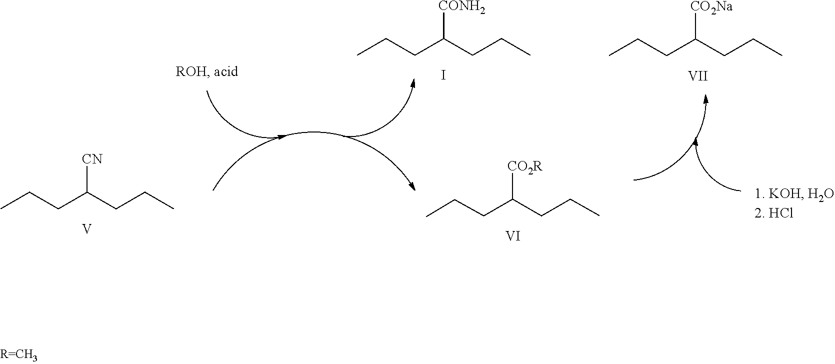

### (1) Preparation of Valpromide

125 g Of propylvaleronitrile and 128 g of methanol, in an ice bath, were dropwise added with 196 g of concentrated sulfuric acid under stirring. The mixture was stirred and reacted at 80° C. for 8 h, then 500 ml of water was added, and the mixture was stirred, adjusted to pH 8 with a sodium hydroxide solution, extracted with ethyl acetate, dried over anhydrous sodium sulfate, subjected to suction filtration, rotary evaporation, and drying to give a solid-liquid mixture. Thereafter, 700 ml of petroleum ether was added, and the mixture was stirred for 0.5 h, left to stand overnight, followed by suction filtration, and washing with petroleum ether. The filtrate was treated according to step (2). The residue was dried to give 34 g of valpromide as a white solid, with a yield of 23.7% (based on propylvaleronitrile), and a melting point of 125.5-126° C. ¹H NMR (400 MHZ, DMSO-d₆) δ: 7.26 (s, 1H, CONH₂), 6.71 (s, 1H, CONH₂), 2.18-2.10 (m, 1H, CH), 1.47-1.36 (m, 2H, CH₂), 1.28-1.17 (m, 6H, CH₂+CH₂×2), 0.85 (t, J=5.8 Hz, 6H, CH₃×2).

### (2) Preparation of Valproic Acid

The filtrate in (1) was subjected to rotary evaporation to recover petroleum ether. Into yellowish liquid, a potassium hydroxide aqueous solution (KOH: 120 g, H₂O: 200 g) was added dropwise, and the mixture was heated to 85° C. and hydrolyzed for 5 h by stirring. The resultant was cooled, and a water layer was separated. The organic phase was added with 350 ml of water, and left to stand for layering to separate an oil phase, and recover propylvaleronitrile. The aqueous phase was adjusted to pH 1 by adding hydrochloric acid and left to stand for layering. The oil phase was dried and rectificated under a reduced pressure to collect 84 g of 85-90° C./0.4 kPa fraction valproic acid, with a yield of 58.3% (based on propylvaleronitrile). ¹H NMR (400 MHz, DMSO-d₆) δ: 11.99 (s, 1H, COOH), 2.24-2.18 (m, 1H, CH), 1.53-1.44 (m, 2H, CH₂), 1.39-1.34 (m, 2H, CH₂), 1.32-1.22 (m, 4H, CH₂×2), 0.86 (t, J=7.2 Hz, 6H, CH₃×2).

In the present description, the present disclosure has been described with reference to specific examples thereof. However, various modifications and changes evidently can be made without departing from the spirit and scope of the present disclosure. Therefore, the description should be regarded as illustrative rather than restrictive.

1. A process for the preparation of valpromide of formula I and sodium valproate of formula II which comprises: performing composite catalytic dipropylation on cyanoacetate and 1-chloropropane in the presence of alkali to obtain 2-cyano-2-valproate of formula III; hydrolyzing and deacidifying 2-cyano-2-valproate to give propylvaleronitrile of formula V; alcoholizing propylvaleronitrile in the presence of acid to give valpromide of formula I and valproate ester of formula VI; and hydrolyzing valproate ester in a sodium hydroxide solution to afford sodium valproate of formula II, wherein the process thereof is as follows: 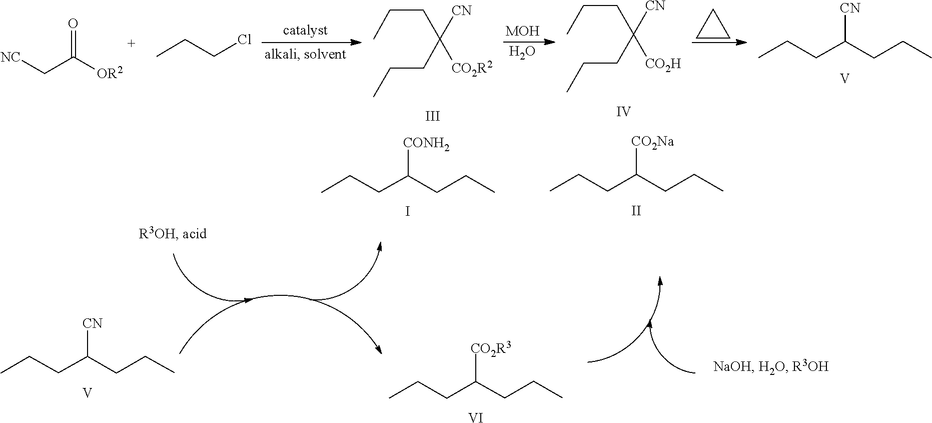
wherein R² is methyl, ethyl, C3-C5 linear alkyl, or C3-C5 branched alkyl; and R³ is methyl or ethyl;
the catalyst for the preparation of 2-cyano-2-valproate of formula III consists of catalyst A and catalyst B;
the catalyst A is R₃N, PhNR₂, R₄NX, or R₃R¹NX, wherein R is C1-C4 linear alkyl or C5-C8 linear alkyl; R¹ is PhCH₂, C1-C5 linear alkyl, or C6-C18 linear alkyl; and X is Cl, Br, or I; and the catalyst B is NaBr, KBr, NaI, or KI;
the solvent for the preparation of 2-cyano-2-valproate of formula III is one or two selected from the group consisting of THE, DMF, DMC, DMSO, acetonitrile, propionitrile, butyronitrile, 1,4-dioxane, ethylene glycol dimethyl ether, ethylene glycol diethyl ether, diethylene glycol dimethyl ether, diethylene glycol diethyl ether, ethyl acetate and butyl acetate;
the alkali for the preparation of 2-cyano-2-valproate of formula III is granular M₂CO₃ or powdery M₂CO₃, wherein M is Na or K;
the acid for an alcoholysis reaction of propylvaleronitrile of formula V is hydrogen chloride gas, aluminum trichloride, thionyl chloride, trifluoromethanesulfonic acid, trimethylsilyl trifluoromethanesulfonate, methanesulfonic acid, benzenesulfonic acid, p-toluenesulfonic acid, or sulfuric acid.

2. The process according to claim 1, wherein R₄NX is tetrabutylammonium chloride, tetrabutylammonium bromide, tetrabutylammonium iodide, tetraethylammonium chloride, tetraethylammonium bromide, tetraethylammonium iodide, tetramethylammonium chloride, tetramethylammonium bromide, tetramethylammonium iodide, or tetrapropylammonium bromide; and
R₃R¹NX is hexadecyltrimethylammonium bromide, octadecyltrimethylammonium bromide, benzyltriethylammonium chloride, b benzyltrimethylammonium chloride, benzyltriethyl-ammonium bromide, hexadecyltriethylammonium bromide, dodecyltriethylammonium bromide, decyltriethylammonium bromide, octyltriethylammonium bromide, hexyltriethylammonium bromide, or trioctylmethylammonium chloride; and
R₃N is trimethylamine, triethylamine, tripropylamine and tributylamine; and PhNR₂ is selected from the group consisting of N,N-dimethylaniline, N,N-diethylaniline, N,N-dipropyl-aniline, or N,N-dibutylaniline.

3. The process according to claim 1, wherein the powdery M₂CO₃ is 100-mesh M₂CO₃, 150-mesh M₂CO₃, 200-mesh M₂CO₃, 250-mesh M₂CO₃, 300-mesh M₂CO₃, or 350-mesh M₂CO₃, wherein M is Na or K.

4. The process according to claim 1, wherein the reaction to form the 2-cyano-2-valproate of formula III is conducted in a temperature of 60-120° C.; and the reaction time is in the range of 1.0-12 h.

5. The process according to claim 1, wherein the reaction to form the 2-cyano-2-valproate of formula III is carried out at a molar ratio of cyanoacetate, the catalyst A, and the catalyst B in the range of 1:0.01-0.10:0.005-0.05; and cyanoacetate is methyl cyanoacetate, ethyl cyanoacetate, n-propyl cyanoacetate, isopropyl cyanoacetate, n-butyl cyanoacetate, or t-butyl cyanoacetate.

6. The process according to claim 1, wherein the alcoholysis reaction of propylvaleronitrile of formula V is carried out at a molar ratio of propylvaleronitrile to acid in the range of 1:1.2-4; the mass concentration of the acid is in the range of 30-70%; and the molar ratio of propylvaleronitrile to R³OH is in the range of 1:3-8.

7. The process according to claim 1, wherein the alcoholysis reaction of propylvaleronitrile of formula V is conducted in a temperature of 25-100° C.; and the alcoholysis reaction time is in the range of 4-24 h.

8. The process according to claim 1, wherein propylvaleronitrile of formula V is alcoholized to prepare valpromide of formula I and valproate ester of formula VI; and the valproate ester is hydrolyzed in the sodium hydroxide solution to give sodium valproate of formula II, wherein the molar ratio of I to II is in the range of 1:1.5-8.0.

9. A process for co-producing valpromide of formula I and valproic acid of formula VII, comprising preparing propylvaleronitrile of formula V according to claim 1; and performing alcoholysis and hydrolysis on propylvaleronitrile to prepare valpromide of formula I and valproic acid of formula VII, wherein the process thereof is as follows: 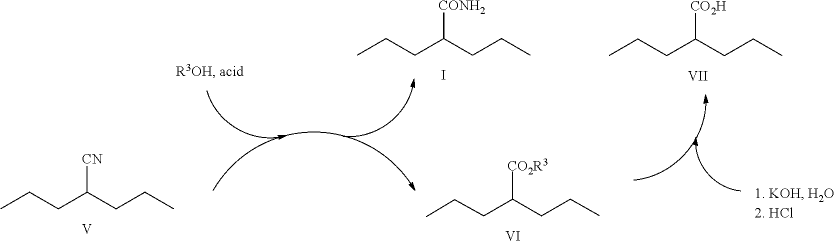
wherein R³ and acid are defined as those in claim 1; and a molar ratio of I to VII is in the range of 1:1.5-8.0.

10. The process according to claim 9, wherein R₄NX is tetrabutylammonium chloride, tetrabutylammonium bromide, tetrabutylammonium iodide, tetraethylammonium chloride, tetraethylammonium bromide, tetraethylammonium iodide, tetramethylammonium chloride, tetramethylammonium bromide, tetramethylammonium iodide, or tetrapropylammonium bromide; and
R₃R₁NX is hexadecyltrimethylammonium bromide, octadecyltrimethylammonium bromide, benzyltriethylammonium chloride, benzyltrimethylammonium chloride, benzyltriethyl-ammonium bromide, hexadecyltriethylammonium bromide, dodecyltriethylammonium bromide, decyltriethylammonium bromide, octyltriethylammonium bromide, hexyltriethylammonium bromide, or trioctylmethylammonium chloride; and
R₃N is trimethylamine, triethylamine, tripropylamine and tributylamine; and PhNR₂ is selected from the group consisting of N,N-dimethylaniline, N,N-diethylaniline, N,N-dipropyl-aniline, or N,N-dibutylaniline.

11. The process according to claim 9, wherein the powdery M₂CO₃ is 100-mesh M₂CO₃, 150-mesh M₂CO₃, 200-mesh M₂CO₃, 250-mesh M₂CO₃, 300-mesh M₂CO₃, or 350-mesh M₂CO₃, wherein M is Na or K.

12. The process according to claim 9, wherein the reaction to form the 2-cyano-2-valproate of formula III is conducted in a temperature of 60-120° C.; and the reaction time is in the range of 1.0-12 h.

13. The process according to claim 9, wherein the reaction to form the 2-cyano-2-valproate of formula III is carried out at a molar ratio of cyanoacetate, the catalyst A, and the catalyst B in the range of 1:0.01-0.10:0.005-0.05; and cyanoacetate is methyl cyanoacetate, ethyl cyanoacetate, n-propyl cyanoacetate, isopropyl cyanoacetate, n-butyl cyanoacetate, or t-butyl cyanoacetate.
